(19) United States
(12) Patent Application Publication
(10) Pub. No.: US 2018/0296600 A1
(43) Pub. Date: Oct. 18, 2018

(54) TREATMENT OF EPIDERMOLYSIS BULLOSA BY INJECTION OF AUTOLOGOUS COLLAGEN VII OVEREXPRESSING LEUKOCYTES

(71) Applicant: The Board of Trustees of the Leland Stanford Junior University, Stanford, CA (US)

(72) Inventor: M. Peter Marinkovich, Redwood City, CA (US)

(21) Appl. No.: 15/766,936

(22) PCT Filed: Oct. 27, 2016
(86) PCT No.: PCT/US16/59172

§ 371 Date: Apr. 9, 2018

Related U.S. Application Data
(60) Provisional application No. 62/247,000, filed on Oct. 27, 2015.

(51) Int. Cl. A61K 35/17; A61K 38/39; A61P 17/02; C12N 5/0781; C07K 14/78; C12N 9/02

(52) U.S. Cl. CPC A61K 35/17; A61K 38/39; A61P 17/02; C12N 2510/00; C07K 14/78; C12N 9/0071; C12Y 114/11002; C12N 5/0635

(57) ABSTRACT

Methods are provided for the cell-based delivery of collagen VII for the treatment of epidermolysis bullosa.

### CROSS REFERENCE

This application claims benefit of U.S. Provisional Patent Application No. 62/247,000, filed Oct. 27, 2015, which application is incorporated herein by reference in its entirety.

### INTRODUCTION

Recessive Dystrophic Epidermolysis Bullosa (RDEB) is an inherited genetic blistering skin disorder caused by mutations in the COL7A1 gene (collagen VII, C7) leading to lack of C7 function. Patients with this disorder are characterized by widespread blistering and erosions of the skin and mucosal tissues including oropharynx, conjuncitivae, esophagus, as well as distal aspects of the genitourinary and gastrointestinal tract. Painful blistering and erosions are a major disability, however scarring from healed wounds also causes significant morbidity, including mitten hand deformities (pseudosyndactyly) of the hands, symblepharon of the eyes, esophageal structures, microstomia, ankyloglossia and strictures of the limbs. It is known that chronic wounding and scarring predisposes to invasive squamous cell carcinoma invasion, and this is a serious problem in RDEB, with invasive squamous cell carcinoma being the leading cause of death in this population starting from the second decade. Therefore, an optimal therapy for this disease would be one which could be implemented early to prevent disabling scarring from occurring, as well as preventing blistering. Also the ability to systemically correct both skin and mucosal tissues would be highly desirable in an RDEB therapeutic approach.

Type VII collagen, (C7) is a large homotrimeric triple helical collagenous molecule, which undergoes anti-parallel dimer formation at its NC2 end, followed by supramolecular assembly into attachment structures termed anchoring fibrils, which connect the lamina densa of the BMZ to the papillary dermis. C7 contains a large NC1 domain, which binds laminin-332 in the lamina densa and a collagenous domain, which wraps around interstitial collagen fibrils in the papillary dermis. Thus, lack of C7 in RDEB produces blistering between the papillary dermis and lamina densa.

Despite advances in the molecular diagnosis of this disease, current therapy is limited to palliative care. While several approaches have been proposed to replace C7, all have their limitations. Topically applied rC7 cannot penetrate intact skin, and is limited to wounded areas. Intradermal rC7 protein injections for RDEB patients are another alternative, however limited diffusion from conventional needle injection necessitates rC7 microneedle array delivery, which is not yet available for clinical use.

Intradermal injection of genetically engineered fibroblasts has also shown promise but suffers from limitation of diffusion from injected sites. Intravenous injection of rC7 is currently in development as an RDEB therapy, however it remains to be shown how expensive or efficient this process is in delivering C7 to the skin, and the mechanism of transfer of C7 from the circulation to wounded skin remains unclear. C7 overexpressing keratinocyte autografts transplanted to human RDEB patients have been successful in reversing blistering, however technical obstacles currently limit this approach to cutaneous sites. Bone marrow replacement strategies have unfortunately been plagued with high mortality rates exceeding that of untreated RDEB patients and long-term follow-up is lacking.

For therapeutic purposes, delivery of C7 is desirable. The present invention addresses this issue.

### SUMMARY OF THE INVENTION

Compositions and methods are provided for the treatment of epidermolysis bullosa in a subject. In the treatment methods of the invention, a population of leukocytes is engineered to over-express C7. Included in the invention is an isolated population of leukocytes engineered to over-express C7, which may be provided in a pharmaceutical unit dose composition. In some embodiments the subject is a human. In some embodiments, the subject is a human suffering from a genetic defect in C7, causing the EB. In the embodiments the genetic defect is Recessive Dystrophic Epidermolysis Bullosa (RDEB). In other embodiments, the subject is an animal model for RDEB, including without limitation an immunodeficient mouse xenografted with keratinocyte/fibroblast containing skin equivalents. Animal models find use, for example, in pre-clinical testing, determining effective dosage of cells, and the like.

In some embodiments, the leukocytes utilized in treatment are autologous peripheral blood mononuclear cells. In such embodiments, the cells may be engineered through introduction of mRNA or transient expression vectors to over-produce C7, usually mRNA, which is introduced through any convenient method, including without limitation electroporation.

In other embodiments, the leukocytes utilized in treatment are plasmablasts, which are stably engineered for persistent expression of C7. In some embodiments the cells are engineered ex vivo, and returned to the subject for therapy. Methods of ex vivo engineering may be selected from, without limitation, virus-free integrative methods, which include transposons, mini-circle integration, CRISPR/Cas9 genome editing system, and the like. In some embodiments a minimal backbone vector is used to reduce the size of the construct, e.g. a mini-circle vector, etc. Optionally the plasmablasts are additionally engineered for persistent expression of prolyl-4-hydroxylase.

In some embodiments of the invention, a method is provided for treatment of EB, the method comprising obtaining a population of leukocytes from a subject suffering from EB, modifying the leukocytes to over-express C7, and reintroducing the leukocytes into the individual.

In another embodiment, the invention provides a composition comprising an electroloaded PBMC transiently expressing C7 at a dose effective to reduce the symptoms of EB, and a pharmaceutically acceptable carrier. In one aspect of the invention, the composition is frozen. In certain embodiments, the composition is free or substantially free of viral vectors and viral-like particles. In some embodiments, the PBMC are autologous relative to an individual selected for treatment.

In another embodiment, the invention provides a composition comprising an a population of plasmoblasts genetically modified to express C7 at a dose effective to reduce the symptoms of EB, and a pharmaceutically acceptable carrier. In one aspect of the invention, the composition is frozen. In certain embodiments, the composition is free or substantially free of viral vectors and viral-like particles. In some embodiments, the plasmoblasts are autologous relative to an individual selected for treatment.

### BRIEF DESCRIPTION OF THE DRAWINGS

FIG. 1. A cell survey of prolyl hydroxylase 4 expression. Lysate samples from indicated cells was equalized for total protein loading and analyzed by immunoblot using prolyl 4 hydroxylase Ab.

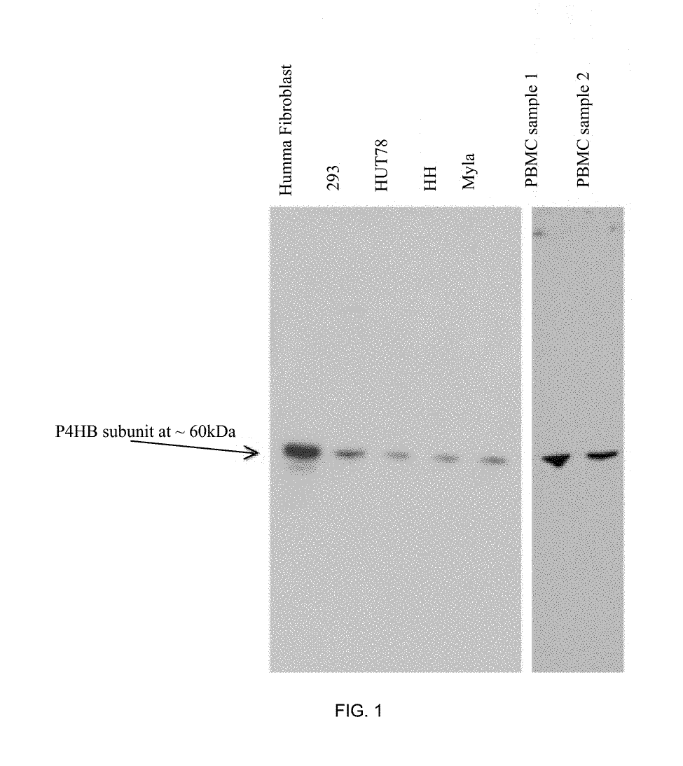

FIG. 2. Overexpression of type VII collagen (C7) in human CD19+plasmablast cultures. Full length COL7A1 cDNA was electroporated into four separate human CD19+ plasmablast cultures (PB1-4). Following 24 hours of culture, conditioned medium was obtained from each transfected plasmablast culture, from non-transfected (control) plasmablasts (CPB), or from normal human keratinocyte cultures (NHK) and analyzed by Western blot using human type VII collagen polyclonal antibody. Molecular weight markers are shown to the left (in kD). Position of full length C7 is indicated to the left (C7). Note keratinocyte medium was concentrated 100 fold prior to loading on Western blot while plasmablast culture medium was loaded onto Western blot unconcentrated.

### DESCRIPTION OF THE SPECIFIC EMBODIMENTS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Any embodiment of any of the present methods, devices, and systems may consist of or consist essentially of, rather than comprise/include/contain/have, the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Conditions of interest for treatment with engineered leukocytes of the present invention include, without limitation, various forms of epidermolysis bullosa, including acquired and congenital forms, the latter of which may be recessive or dominant.

Based on the most recent classification system, dystrophic epidermolysis bullosa (DEB) includes three subtypes: recessive DEB, severe generalized (RDEB-sev gen) (formerly called Hallopeau-Siemens type (RDEB-HS); recessive DEB, generalized other (RDEB-O) (formerly called non-Hallopeau-Siemens type (RDEB-non-HS); and dominant DEB (DDEB). In RDEB-sev gen, blisters affecting the whole body may be present in the neonatal period. Oral involvement may lead to mouth blistering, fusion of the tongue to the floor of the mouth, and progressive diminution of the size of the oral cavity. Esophageal erosions can lead to webs and strictures that can cause severe dysphagia. Consequently, severe nutritional deficiency and secondary problems are common. Corneal erosions can lead to scarring and loss of vision. Blistering of the hands and feet followed by scarring fuses the digits into “mitten” hands and feet, a hallmark of this disorder. The lifetime risk of aggressive squamous cell carcinoma is over 90%. In DDEB, blistering is often mild and limited to hands, feet, knees, and elbows, but nonetheless heals with scarring. Dystrophic nails, especially toenails, are common and may be the only manifestation of DDEB.

Conventional treatment of manifestations is primarily supportive, including wound dressing and nutritional support. Occupational therapy may help prevent hand contractures. Surgical release of fingers often needs to be repeated.

Leukocytes engineered to over-express C7 can find use in therapy for dystrophic epidermolysis bullosa

In addition to inherited forms of EB, the acquired form of epidermolysis bullosa (EBA) involves pathology in type VII collagen and may be treated with the engineered leukocytes of the invention. Circulating autoantibodies in patients with EBA recognize epitopes in type VII collagen molecules, and molecular cloning of the type VII collagen cDNAs have provided the tools to identify the most predominant immunoepitopes within the amino-terminal NC-1 domain of type VII collagen. The antigenic properties of the NC-1(VII) domain are further highlighted by the fact that monoclonal antibodies, such as H3A and L3D, which are in clinical use to map type VII collagen in the skin of patients with inherited forms of EB, also identify epitopes in this portion of the protein. In addition to circulating autoantibodies recognizing type VII collagen epitopes in EBA, bullous lesions in some patients with systemic lupus erythematosus have also been associated with anti-type VII collagen antibodies.

Collagen. As used herein the term “collagen” refers to compositions in which at least about 50%, at least about 60%, at least about 70%, at least about 80%, at least about 90%, at least about 95% or more of the protein present is collagen in a triple helical configuration. The folding of the individual α-chains into the triple-helical conformation is predicated upon the characteristic primary sequence, consisting of repeating Gly-X-Y triplet sequences. Collagens are widely found in vertebrate species, and have been sequenced for many different species. Due to the high degree of sequence similarity between species, collagen from different species can be used for biomedical purposes, e.g. between mammalian species, although the human protein may be preferred

FACIT collagens (fibril-associated collagens with interrupted triple helices) include types IX, XII, XIV, XIX, XX, and XXI. Several of the latter types of collagens associate with larger collagen fibers and serve as molecular bridges, stabilizing the organization of the extracellular matrix. Collagen VII, (COL7A1, Chromosome 3, NC_000003.10 (48576510 . . . 48607689, complement)) is of particular interest. Type VII collagen is a major component of anchoring fibrils.

Type VII collagen is a long, 424 nm, triple-helical domain with flanking non-collagenous sequences. Type VII collagen molecules consists of a central collagenous, triple-helical segment flanked by the non-collagenous NC-1 and NC-2 domains. Unlike interstitial collagens, the repeating Gly-X-Y sequence is interrupted by 19 imperfections due to insertions or deletions of amino acids in the Gly-X-Y repeat sequence. Most notably, in the middle of the triple-helical domain, there is a 39-amino acid non-collagenous “hinge” region which is susceptible to proteolytic digestion with pepsin. The amino-terminal NC-1 domain of type VII, approximately 145 kDa in size, consists of sub-modules with homology to known adhesive proteins, including segments with homology to cartilage matrix protein (CMP), nine consecutive fibronectin type III-like (FN-III) domains, a segment with homology to the A domain of von Willebrand factor, and a short cysteine and proline-rich region. The carboxy-terminal non-collageneous domain, NC-2, is relatively small, ˜30 kDa, and it contains a segment with homology to Kunitz protease inhibitor molecule.

The human type VII collagen gene, COL7A1 has a complex structure consisting of a total of 118 separate exons. The gene is, however, relatively compact, and most of the introns are relatively small; consequently, the size of the entire human COL7A1 gene is only ˜32 kb, encoding a messenger RNA of ˜8.9 kb. COL7A1 has been mapped to the short-arm of human chromosome 3, region 3p21.1. The type VII collagen gene structure and the encoded primary sequence of the protein are well conserved, and for example, the mouse gene shows 84.7 percent homology at the nucleotide and 90.4 percent identity at the protein level.

Type VII collagen is synthesized both by epidermal keratinocytes and dermal fibroblasts in culture. Upon synthesis of complete pro-α1(VII) polypeptides, three polypeptides associate through their carboxy-terminal ends to a trimer molecule which in its collagenous portion folds into the triple-helical formation. The triple-helical molecules are then secreted to the extracellular milieu where two type VII collagen molecules align into an anti-parallel dimer with the amino-terminal domains present at both ends of the molecule. This dimer assembly is accompanied by proteolytic removal of a portion of the carboxy-terminal end of both type VII collagen molecules and stabilization by inter-molecular disulfide bond formation. Subsequently, a large number of these anti-parallel dimers aggregate laterally to form anchoring fibrils.

Glycine substitution mutations in the triple helical domain of COL7A (especially in exons 73, 74, and 75) predominate in dominant dystrophic epidermolysis bullosa (DDEB). Mutations p.Gly2034Arg and p.Gly2043Arg are the most common DDEB-causing mutations, making up 50% of the dominant mutations reported in the largest US cohort. Glycine substitutions as well as other amino acid substitutions and splice junction mutations outside of this region may also be found in dominant DEB.

More than 400 recessive DEB-causing mutations spanning the entire gene have been described for all forms of DEB. Each mutation, however, accounts for no more than 1%-2% of the total number of mutations described. Null mutations predominate in RDEB, though glycine substitutions and other amino acid substitutions have been described. Milder forms of RDEB are often caused by splice junction mutations or other missense mutations.

A “native sequence” polypeptide is one that has the same amino acid sequence as a polypeptide derived from nature. Such native sequence polypeptides can produced by recombinant means according to the methods set forth herein. Thus, a native sequence polypeptide can have the amino acid sequence of, e.g. naturally occurring human polypeptide, murine polypeptide, or polypeptide from any other mammalian species, and the like. The term “native sequence collagen VII protein” includes the native proteins with or without the initiating N-terminal methionine (Met).

A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active collagen VII variant will have an amino acid sequence having at least about 90% amino acid sequence identity with a native sequence collagen VII polypeptide, preferably at least about 95%, more preferably at least about 99%.

A “functional derivative” of a native sequence collagen VII polypeptide is a compound having a qualitative biological property in common with a native sequence collagen VII polypeptide. “Functional derivatives” include, but are not limited to, fragments of a native sequence and derivatives of a native sequence collagen VII polypeptide and its fragments, provided that they have a biological activity in common with a corresponding native sequence collagen VII polypeptide. The term “derivative” encompasses both amino acid sequence variants of collagen VII polypeptide and covalent modifications thereof.

Prolyl 4-hydroxylase (EC 1.14.11.2) plays a central role in collagen synthesis. It catalyzes the formation of 4-hydroxyproline in collagens by hydroxylation of proline residues in peptide linkages. The 4-hydroxyproline residues are essential for the folding of the newly synthesized procollagen polypeptide chain into triple helical molecules. The active enzyme is a tetramer of 2 alpha and 2 beta subunits with a molecular weight of about 240,000. The beta subunit (P4HB) is identical to the enzyme disulfide isomerase (EC 5.3.4.1) and a major cellular thyroid-binding protein. The alpha subunit contributes a major part of the catalytic site of the enzyme. The polypeptide is 517 amino acid residues and a signal peptide of 17 amino acids.

The P4HA gene covers more than 69 kilobases and consists of 16 exons. Evidence had previously been presented for a mutually exclusive alternative splicing of RNA transcripts of the gene. The present data indicated that the mutually exclusive sequences found in the mRNAs are coded by 2 consecutive, homologous 71-bp exons, 9 and 10. These exons are identical in their first 5 base pairs and the overall identity between them is 61% at the nucleotide level and 58% at the level of the coded amino acids. Both types of mRNA were found to be expressed in all of the tissues studied, but in some tissues the type coding for the exon 9 or exon 10 sequences was more abundant than the other type.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

In the present methods, collagen VII is produced by either in vitro expression and purification of mRNA, for transient expression in leukocytes; or is introduced into a cell population on an integrating, usually non-viral, expression construct. The DNA encoding collagen VII polypeptide may be obtained from any cDNA library prepared from tissue expressing the collagen VII polypeptide mRNA, prepared from various sources. The collagen VII polypeptide-encoding gene may also be obtained from a genomic library or by oligonucleotide synthesis. An alternative means to isolate the gene encoding is to use PCR methodology.

The nucleic acid (e.g., cDNA or genomic DNA) encoding the collagen VII polypeptide is inserted into a construct for expression, operably linked to elements required for expression. Many such constructs are available. The components generally include, but are not limited to, one or more of the following: the coding sequence, one or more marker genes, an enhancer element, a promoter, and a transcription termination sequence.

A “vector” is capable of transferring nucleic acid sequences to target cells. For example, a vector may comprise a coding sequence capable of being expressed in a target cell. For the purposes of the present invention, “vector construct,” “expression vector,” and “gene transfer vector,” generally refer to any nucleic acid construct capable of directing the expression of a gene of interest and which is useful in transferring the gene of interest into target cells. Thus, the term includes cloning and expression vehicles, as well as integrating vectors.

An “expression cassette” comprises any nucleic acid construct capable of directing the expression of any RNA transcript including gene/coding sequence of interest as well as non-translated RNAs, such as shRNAs, microRNAs, siRNAs, anti-sense RNAs, and the like. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adapters or linkers are used in accordance with conventional practice.

Expression vectors will contain a promoter that is recognized by the autologous leukocyte, or a host cell for expression of mRNAs, and is operably linked to the collagen VII coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known. Heterologous promoters are preferred, as they generally permit greater transcription and higher yields.

Transcription from vectors in mammalian host cells may be controlled, for example, by promoters obtained from the genomes of viruses such as polyoma virus, fowlpox virus, adenovirus (such as adenovirus 2), bovine papilloma virus, avian sarcoma virus, cytomegalovirus, a retrovirus, hepatitis-B, simian virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA.

Vectors and systems for non-viral integration of an expression cassette into a cell are known in the art, and may include, without limitation, transposon systems, CRISPR/Cas9 systems, and minicircle vectors.

Minicircle Vector. As used herein a minicircle vector is a small, double stranded circular DNA molecule that provides for persistent, high level expression of a sequence of interest that is present on the vector. For the purposes of the present invention the sequence of interest is a sequence encoding one or a plurality of reprogramming factors. The sequence of interest is operably linked to regulatory sequences present on the mini-circle vector, which regulatory sequences control its expression. Such mini-circle vectors are described, for example in published U.S. Patent Application US20040214329, herein specifically incorporated by reference.

The overall length of the subject minicircle vectors is sufficient to include the desired elements as described below, but not so long as to prevent or substantially inhibit to an unacceptable level the ability of the vector to enter the target cell upon contact with the cell, e.g., via systemic administration to the host comprising the cell. As such, the minicircle vector is generally at least about 0.3 kb long, often at least about 1.0 kb long, where the vector may be as long as 10 kb or longer, but in certain embodiments do not exceed this length.

The effective dose of a minicircle vector for introduction into cells may be empirically determined by one of skill in the art. For example, minicircle vectors may be provided to cells at a concentration of at least about 1 ng for 10⁶ cells, about 10 ng for 10⁶ cells, about 100 ng for 10⁶ cells, about 1 μg for 10⁶ cells, about 5 μg for 10⁶ cells, or more. Typically high concentrations are not deleterious.

Minicircle vectors differ from bacterial plasmid vectors in that they lack an origin of replication, and lack drug selection markers commonly found in bacterial plasmids, e.g. β-lactamase, tet, and the like. Consequently, minicircles are small in size, allowing more efficient delivery to a cell. More importantly, minicircles are devoid of the transgene expression silencing effect which is associated with the vector backbone nucleic acid sequences of parental plasmids from which the minicircle vectors are excised. The minicircle may be substantially free of vector sequences other than the recombinase hybrid product sequence, and the sequence of interest, i.e. a transcribed sequence and regulatory sequences required for expression.

An alternative to mini-circle vectors is the use of a transposon, e.g. the sleeping beauty (SB) transposon system to introduce a C7 expression cassette into a cell. The mobilization of SB elements is a specialized form of DNA recombination and occurs by a cut-and-paste pathway involving a DNA intermediate. This transposition process involves five distinct stages: (i) association of the transposase with its binding sites within the transposon IRs; (ii) assembly of an active synaptic complex in which the two ends of the element are paired and held together by bound transposase subunits; (iii) transposase-mediated excision of the element from its original donor site, (iv) re-insertion of the excised element into a new target site (TA dinucleotide); and (v) repair of the cellular DNA at both the excision and re-insertion sites.

By Sleeping Beauty transposon is meant a nucleic acid that is flanked at either end by inverted repeats which are recognized by an enzyme having Sleeping Beauty transposase activity. By ‘recognized’ is meant that a Sleeping Beauty transposase is capable of binding to the inverted repeat and then integrating the transposon flanked by the inverted repeat into the genome of the target cell. Representative inverted repeats that may be found in the Sleeping Beauty transposons of the subject methods include those disclosed in WO 98/40510 and WO 99/25817. Of particular interest are inverted repeats that are recognized by the wild type Sleeping Beauty transposase. Vectors suitable for the use of SB transposase to integrate DNA into a cell genome are commercially available, e.g. see Discovery Genomics, U.S. Pat. No. 6,489,458. The Sleeping Beauty transposon is generally present on a vector which is introduced into the cell. The transposon may be present on a variety of different vectors, where representative vectors include plasmids, linear DNA molecules and the like.

In many embodiments, each inverted repeat of the transposon includes at least one direct repeat. The transposon element is a linear nucleic acid fragment that can be used as a linear fragment or circularized, for example in a plasmid. In certain embodiments, there are two direct repeats in each inverted repeat sequence.

The sleeping beauty components are introduced into the target cell. Any convenient protocol may be employed, where the protocol may provide for in vitro or in vivo introduction of the system components into the target cell, depending on the location of the target cell. For example, where the target cell is an isolated cell, the system may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

Methods of introducing an expression cassette into the genome of a cell also include CRISPR/Cas systems, for example see Hsu et al. (2014) Cell 157:1262-1278; and Makarova et al. (2015) Nature Reviews Microbiology 13:1-15; each herein specifically incorporated by reference. In these systems, which are commercially available from multiple sources, including ClonTech, Thermo Fisher, etc. In these systems, a guide RNA and a Cas9 protein form a complex. The guide RNA provides target specificity to the complex by comprising a nucleotide sequence that is complementary to a sequence (the target site) of a target DNA. The Cas9 protein of the complex provides the site-specific activity. In other words, the Cas9 protein is guided to a target site within a target DNA (e.g. a chromosomal or extrachromosomal DNA) by virtue of its association with the guide RNA. A Cas9 protein can bind and/or modify (e.g., cleave, methylate, demethylate, etc.) a target DNA and/or a polypeptide associated with a target DNA (e.g., methylation or acetylation of a histone tail). In some cases, the Cas9 protein is a naturally-occurring protein (e.g, naturally occurs in bacterial and/or archaeal cells), e.g., the Cas9 protein can cause a double strand break by cleaving both strands of a target double-stranded DNA. In other cases, the Cas9 protein is not a naturally-occurring polypeptide (e.g., the Cas9 protein can be a variant such as a nickase variant, a catalytically inactive Cas9, a fusion protein, and any combination thereof).

For high efficiency production of mRNA, various vector and expression systems may be used. Frequently bacterial systems are used in these systems due to the ease of culturing the cells, e.g. vectors utilizing the T7, Lac, Trp, etc. promoter systems, which can be provided in a high copy number plasmid. The cap structure and poly(A) tail of eukaryotic mRNA are critical for mRNA stability and translational efficiency; and it is desirable to use a system in which these modifications are included, e.g. see WO 2011/128444; the mScript™ mRNA Production System; etc.

Where the cells are modified by introduction of mRNA encoding C7, the mRNA may be introduced by electroporation. Electroporation is a well recognized method for loading nucleic acids into cells to achieve transfection of the loaded cells. A method of transfecting cells may be referred to as electroloading, where there is no transfecting reagent or biologically based packaging of the nucleic acid being loaded, such as a viral vector or viral-like particle, relying only on a transient electric field being applied to the cell to facilitate loading of the cell.

Loading of cells with mRNA brings several advantages. mRNA, especially when loaded by electroloading results in minimal cell toxicity relative to loading with plasmid DNA, and this is especially true for electroloading of resting cells such as peripheral blood mononuclear cells (PBMC) cells. Since mRNA need not enter the cell nucleus to be expressed resting cells readily express loaded mRNA. Further, since mRNA need not be transported to the nucleus, or transcribed or processed it can begin to be translated essentially immediately following entry into the cell's cytoplasm. This allows for rapid expression of the gene coded by the mRNA. Moreover, mRNA does not replicate or modify the heritable genetic material of cells and mRNA preparations typically contain a single protein coding sequence, which codes for the protein one wishes to have expressed in the loaded cell. Various studies on mRNA electroloading have been reported (Landi et al., 2007; Van De Parre et al. 2005; Rabinovich et al. 2006; Zhao et al., 2006).

Those of skill in the art are familiar with methods of electroporation. The electroporation may be, for example, flow electroporation or static electroporation. In one embodiment, the method of transfecting the cells comprises use of an electroporation device as described in, for example, published PCT Application Nos. WO 03/018751 and WO 2004/031353; U.S. patent application Ser. Nos. 10/781,440, 10/080,272, and 10/675,592; and U.S. Pat. Nos. 5,720,921, 6,074,605, 6,773,669, 6,090,617, 6,485,961, 6,617,154, 5,612,207, all of which are incorporated by reference.

Leukocytes and PBMC. Mononuclear cells, encompassing for example hematopoietic stem cells, mesenchymal stem cells, endothelial progenitor cells, adipose derived stem cells, and peripheral blood mononuclear cells (PBMC), have been used in multiple applications for treatment of immune diseases and in regenerative medicine applications (Passweg J and Tyndall A., Semin Hematol. 2007 October 44(4):278-85; Le Blanc K and Ringdén O. Intern Med. 2007 November 262(5):509-25; Ward et al. Catheter Cardiovasc Interv. 2007 Dec. 1 70(7):983-98; Mimeault et al., Clin Pharmacol Ther. September 2007 82(3):252-64 Epub 2007 Aug. 1). Peripheral blood mononuclear cells (PBMC) are comprised of cells of myeloid and lymphoid lineages. Myeloid cells include monocytes, macrophages, dendritic cells (DC). Lymphoid cells include T cells, NK cells, B cells, lymphoid DC.

Freshly collected primary PBMCs may be collected, isolated, and transfected within about 0.5 to 3 hours, 0.5 to 2 hours, or 0.5 to 1 hour. In some embodiments, the freshly collected primary PBMCs are frozen immediately after being collected from patient. The PBMCs may be frozen in peripheral blood or they may be isolated and then frozen or they may be isolated, transfected and then frozen. Thus, in certain aspects of the invention, fresh primary PBMCs may be thawed cells that were frozen immediately after collection from a patient/donor or immediately after isolation following collection. In some embodiments, the transfected cells are administered to the patient within about 1 to 48 hours, 1 to 24 hours, 1 to 15 hours, 1 to 10 hours, or 1 to 5 hours from the time the cells were originally obtained from the patient or donor.

B Cells and Plasmablasts. In certain embodiments, the leukocytes engineered by the methods of the invention are B cells or plasmablasts, for example US Patent application 20130143267, herein specifically incorporated by reference. Prior to genetic alteration and differentiation, a source of B cells is obtained from a subject. B cells can be obtained from a number of sources, including peripheral blood mononuclear cells, bone marrow, lymph node tissue, cord blood, tissue from a site of infection, spleen tissue, and tumors. B cells can be obtained from a unit of blood collected from a subject using any number of techniques known to the skilled artisan. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. After washing, the cells may be resuspended in a variety of biocompatible buffers, such as PBS, normal saline, media, and the like.

B cells may be isolated from peripheral blood or leukapheresis using techniques known in the art. For example, PBMCs may be isolated using FICOLL™ (Sigma-Aldrich, St Louis, Mo.) and CD19+B cells purified by negative or positive selection using any of a variety of antibodies known in the art, such as the Rosette tetrameric complex system (StemCell Technologies, Vancouver, Canada). Other isolation kits are commercially available, such as R&D Systems' MagCellect Human B Cell Isolation Kit (Minneapolis, Minn.), or isolation may be performed by flow cytometry.

Resting B cells may be prepared by sedimentation on discontinuous Percoll gradients, as described in (Defranco et al., (1982) J. Exp. Med. 155:1523). In brief, cells isolated from the 70-75% (density of 1.087-1.097) Percoll interface are typically >95% mlg.sup.+, have a uniform, low degree of near forward light scatter and are unresponsive to Con A.

Genetically altered B cells may be cultured so as to promote differentiation and activation such that the B cells actively produce the transgene-encoded C7 protein, and if required, transgene-encoded prolyl-4-hydroxylase. In this regard, the B cells are activated and differentiate into plasma cells. As would be recognized by the skilled person, plasma cells may be identified by cell surface protein expression patterns using standard flow cytometry methods. For example, terminally differentiated plasma cells express relatively few surface antigens, and do not express common pan-B cell markers, such as CD19 and CD20. Instead, plasma cells are identified through flow cytometry by their additional expression of CD38, CD78, the Interleukin-6 receptor and lack of expression of CD45. In humans, CD27 is a good marker for plasma cells, naive B cells are CD27-, memory B-cells are CD27+and plasma cells are CD27++. CD38 and CD138 are expressed at high levels.

In certain embodiments, it may be desirable to differentiate and activate B cells prior to genetic modification.

In one embodiment, the B cells may be contacted with a B cell activating factor, e.g., any of a variety of cytokines, growth factors or cell lines known to activate and/or differentiate B cells (see e.g., Fluckiger, et al. Blood 1998 92: 4509-4520; Luo, et al., Blood 2009 113: 1422-1431). Such factors may be selected from the group consisting of, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35, IFN-γ, IFN-α, IFN-β, IFN-ω, C type chemokines XCL1 and XCL2, C-C type chemokines (to date including CCL1-CCL28) and CXC type chemokines (to date including CXCL1-CXCL17), and members of the TNF superfamily (e.g., TNF-α, 4-1BB ligand, B cell activating factor (BLyS), FAS ligand, Lymphotoxin, OX4OL RANKL and TRAIL). In particular, genetically altered B cells (or B cells prior to transduction) may be contacted or cultured on feeder cells. In certain embodiments, the feeder cells are a stromal cell line, e.g., the murine stromal cell lines S17 or MS5. In a further embodiment, purified CD19+cells may be cultured in the presence of fibroblasts expressing CD40-ligand in the presence of B cell activating factor cytokines such as IL-10 and IL-4. CD4OL may also be provided bound to a surface such as tissue culture plate or a bead. In another embodiment, purified CD19+cells may be cultured in the presence of feeder cells expressing CD40L in presence of one or more cytokines or factors selected from IL-10, IL-4, IL-7, CpG DNA, IL-2, IL-15, IL6, and IFN-α.

Contact of the B cells with B cell activation factors leads to, among other things, cell proliferation, modulation of the IgM⁺ cell surface phenotype to one consistent with an activated mature B cell, secretion of Ig, and isotype switching. CD19⁺ B cells may be isolated using known and commercially available cell separation kits, such as the MiniMacs cell separation system (Miltenyi Biotech, Bergisch Gladbach, Germany). In certain embodiments, CD40L fibroblasts are irradiated before use in the methods described herein.

In a further embodiment, progenitor cells or B cells may be cultured in the presence of one or more of IL-3, IL-7, Flt3 ligand, thrombopoietin, SCF, IL-2, IL-10, G-CSF and CpG. In certain embodiments, the methods include culturing the B cells or progenitors in the presence of one or more of the aforementioned factors in conjunction with transformed stromal cells (e.g., MS5) providing a low level of anchored CD40L, or CD40L bound to a plate or a bead.

Any of a variety of culture media may be used in the present methods as would be known to the skilled person (see e.g., Current Protocols in Cell Culture, 2000-2009 by John Wiley & Sons, Inc.). In one embodiment, media for use in the methods described herein includes, but is not limited to Iscove modified Dulbecco medium (with or without fetal bovine or other appropriate serum). Illustrative media also includes, but is not limited to, RPMI 1640, AIM-V, DMEM, MEM, a-MEM, F-12, X-Vivo 15, and X-Vivo 20. In further embodiments, the medium may comprise a surfactant, an antibody, plasmanate or a reducing agent (e.g. N-acetyl-cysteine, 2-mercaptoethanol), or one or more antibiotics. In some embodiments, IL-6, soluble CD40L, and a cross-linking enhancer may also be used.

B cells or progenitor cells may be cultured under conditions and for sufficient time periods to achieve differentiation and activation desired. In certain embodiments, the B cells or progenitor cells are cultured under conditions and for sufficient time periods such that 10%, 15%, 20%, 25%, 30%, 35%, 40%, 45%, 50%, 55%, 60%, 65%, 70%, 75%, 80%, 85%, 90%, 95%, or even 100% of the B cells are differentiated and/or activated as desired. In one embodiment, the B cells are activated and differentiate into plasma cells. As would be recognized by the skilled person, plasma cells may be identified by cell surface protein expression patterns using standard flow cytometry methods as described elsewhere herein, such as by expression of CD38, CD78, the Interleukin-6 receptor, CD27^{high} CD138, and lack of expression of common pan-B cell markers, such as CD19 and CD20 and lack of expression of CD45.

In certain embodiments, cells are cultured for 1-7 days. In further embodiments, cells are cultured 7, 14, 21 days or longer. Thus, cells may be cultured under appropriate conditions for 1, 2, 3, 4, 5, 6, 7, 8, 9, 10, 11, 12, 13, 14, 15, 16, 17, 18, 19, 20, 21, 22, 23, 24, 25, 26, 27, 28, 29, or more days. Cells are replated, media and supplements may be added or changed as needed using techniques known in the art.

In certain embodiments, the genetically altered B cells or progenitor cells may be cultured under conditions and for sufficient time periods such that at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the cells are differentiated and activated to produce Ig and/or to express the C7 transgene.

The induction of B cell activation may be measured by techniques such as ³H-uridine incorporation into RNA (as B cells differentiate, RNA synthesis increases), or by ³H-thymidine incorporation, which measures DNA synthesis associated with cell proliferation. For optimal measurement of B cell proliferation, interleukin-4 (IL-4) may be added to the culture medium at an appropriate concentration, such as about 10 ng/ml.

Alternatively, B cell activation may be measured as a function of immunoglobulin secretion. For example, CD4OL may be added to resting B cells together with IL-4 (e.g., 10 ng/ml) and IL-5 (e.g., 5 ng/ml) or other cytokines suited to activation of B cells. Flow cytometry may also be used for measuring cell surface markers typical of activated B cells.

After culture for an appropriate period of time, such as from 2, 3, 4, 5, 6, 7, 8, 9, or more days, generally around 3 days, an additional volume of culture medium may be added. Supernatant from individual cultures may be harvested at various times during culture and quantitated for IgM and IG.sub.1 as described in Noelle et al., (1991) J. Immunol. 146:1118-1124. In further embodiments, the cultures may be harvested and measured for expression of the transgene of interest using flow cytometry, ELISA, ELISPOT or other assay known in the art.

In further embodiments, enzyme-linked immunoadsorption assay (ELISA) may be used for measuring IgM or other antibody isotype production or for production of the transgene of interest. In certain embodiments, IgG determinations may be made using commercially available antibodies such as goat antihuman IgG, as capture antibody, followed by detection using any of a variety of appropriate detection reagents such as biotinylated goat antihuman Ig, streptavidin alkaline phosphatase and substrate.

In one embodiment, the cell compositions of the present disclosure comprise a genetically altered and activated/differentiated B cell population or PBMC population, expressing a human C7 protein in an amount effective for the treatment of EB. In one embodiment, the compositions comprise genetically altered B cells that have differentiated into plasma B cells. Target cell populations, such as the genetically altered and activated B cell populations of the present disclosure may be administered either alone, or as a pharmaceutical composition in combination with diluents and/or with other components such as cytokines or cell populations. Cell compositions may comprise a genetically altered and activated/differentiated B cell population expressing human C7 protein as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Cell compositions of the present disclosure are administered in a manner appropriate to the treatment of EB. The quantity and frequency of administration will be determined by such factors as the condition of the patient, and the type and severity of the patient's disease, although appropriate dosages may be determined by clinical trials.

The cells may be administered to the subject by methods well known to those of skill in the art. For example, the cells may be administered by intravenous injection, intraarterial injection, intralymphatic injection, intramuscular injection, intratumoral injection, or subcutaneous injection. A medical practitioner will be able to determine a suitable administration route for a particular subject based, in part, on the type and location of the disease. The transfected cells may be administered locally to a disease site, regionally to a disease site, or systemically. In one embodiment, the cells are administered by intravenous injection. In some embodiments, e.g. where the cells are PBMC, the transfected cells are administered back in to the patient in less than 48 hours, less than 24 hours, or less than 12 hours from the time from when the peripheral blood is collected from the donor. The donor and the subject being treated may be the same person or different people. Thus, in some embodiments the cells are autologous to the subject; and in other embodiments, the cells are allogeneic to the subject.

Pharmaceutical preparations of engineered cells for administration to a subject are contemplated by the present invention. One of ordinary skill in the art would be familiar with techniques for administering cells to a subject. Furthermore, one of ordinary skill in the art would be familiar with techniques and pharmaceutical reagents necessary for preparation of these cell prior to administration to a subject.

In certain embodiments of the present invention, the pharmaceutical preparation will be an aqueous composition that includes the engineered cells that have been modified to over-express C7 and optionally prolyl-4-hydroxylase. In certain embodiments, the transfected cell is prepared using cells that have been obtained from the subject (i.e., autologous cells).

Pharmaceutical compositions of the present invention comprise an effective amount of a solution of the transfected cells in a pharmaceutically acceptable carrier or aqueous medium. As used herein, “pharmaceutical preparation” or “pharmaceutical composition” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the cells, its use in the therapeutic compositions is contemplated. Supplementary active ingredients can also be incorporated into the compositions. For human administration, preparations should meet sterility, pyrogenicity, general safety and purity standards as required by the FDA Center for Biologics.

A person of ordinary skill in the art would be familiar with techniques for generating sterile solutions for injection or application by any other route. Determination of the number of cells to be administered will be made by one of skill in the art. In certain aspects, multiple doses may be administered over a period of days, weeks, months, or year. A subject may receive, for example, 1, 2, 3, 4, 5, 6, 7, 8, 9, 10, 11, 12, 13, 14, 15, 16, 17, 18, 19, or 20 doses.

When “an effective amount”, or “therapeutic amount” is indicated, the precise amount of the compositions of the present disclosure to be administered can be determined by a physician with consideration of individual differences in age, weight, and condition of the patient (subject). It can generally be stated that a cell composition comprising the cells described herein may be administered at a dosage of 10⁴ to 10⁷ cells/kg body weight, usually 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments of the present disclosure, leukocytes that are genetically engineered using the methods described herein, or other methods known in the art, are administered to a patient in conjunction with (e.g. before, simultaneously or following) any number of relevant treatment modalities.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Cell reprogramming of autologous cells as treatment for Recessive Dystrophic Epidermolysis Bullosa (RDEB)

Recessive dystrophic epidermolysis bullosa (RDEB) is a severe blistering disorder caused by deficiency of type VII collagen (C7) at the dermal-epidermal basement membrane zone (BMZ). Provided herein are two alternative clinically viable cell re-programming treatment strategies to restore C7 and reverse RDEB disease progression. First, is a method to gene-modify autologous PBMCs for transient systemic C7 expression, which will deliver C7 to the BMZ and promote anchoring fibril formation for 1-3 months. The studies will complete the requisite in vitro and in vivo testing for regulatory filings to FDA. Second, the Immune System Programming (ISP™) platform is an ex vivo culture system can be used to produce autologous C7 expressing plasmablasts. Long-term engraftment of ISP™ plasmablasts offers sustained systemic delivery of functional C7.

Electroporation-mediated delivery of mRNA encoding VII collagen (C7) into autologous peripheral blood mononuclear cells (PBMCs) for transient systemic expression of C7. To validate stability, expression and function of C7 secreted from PBMCs in vitro, transient expression of C7 mRNA in mouse and human PBMCs was achieved using research grade flow through bulk electroporator. The electroporated PBMC cells are used to achieve correction of RDEB skin equivalent culture. The transient C7 expression and disease correction in murine RDEB human xenograft model is then used to validate the model, by testing in immune-deficient mice xenografted with RDEB skin equivalent following systemic transfer of the C7 protein-secreting mouse PBMCs.

The primary goal of these experiments is to demonstrate cell re-programming of autologous PBMC's for transient, systemic expression C7 molecule assembled into functional C7 trimers forming anchoring fibrils at the dermal epidermal junction (DEJ), enabling disease correction in an RDEB mouse model.

Clinical development of an autologous ISP™ plasmablast cell product is developed for persistent in vivo expression of C7. Initially, the stability, expression, and function of 07 expressing plasmablasts is analyzed in vitro, To measure C7 expression from ISP™ plasmablasts, blood samples are collected from healthy donors and RDEB patients. RDEB skin equivalent culture correction is assessed following treatment with C7 expressing plasmablasts. Persistent C7 expression and disease correction in murine RDEB human xenograft model is used for validation of the methos. Testing is performed in immunodeficient mice xenografted with RDEB skin equivalents following systemic injection of the C7 expressing plasmablasts,

The cell re-programming strategies develop a cell-based approach for transient and sustained in vivo protein replacement therapy with C7 to treat RDEB. Electroporation-mediated delivery of mRNA encoding C7 into PBMCs is a cell re-programming strategy that results in a transient cell product capable of pulsing high concentrations of C7 systemically as a near-term strategy. Each treatment of autologous PBMCs is estimated to last 1-3 months, and intends to greatly ameliorate symptoms and reduce the suffering experienced by RDEB patients.

The Immune System Programming (ISP™) platform is an ex vivo culture system used to produce an autologous C7 expressing plasmablasts (a.k.a. ISP™ plasmablasts) for persistent in vivo expression of a functional C7 protein—protein capable of assembling into functional anchoring fibrils at the DEJ. By facilitating sustained, systemic delivery of functional C7 for months or years via long-term engraftment of ISP™ plasmablasts, the ISP approach offers a novel strategy and functional cure for RDEB.

Lysates from PBMC samples were blotted with a prolyl-4-hydroxylase (P4H) antibody, all PBMC samples showed good levels of expression relative to a panel of other mammalian cells. In particular, the levels looked only slightly less than fibroblasts, which are well known for their robust posttranslational collagen modifying capability. P4H is essential for C7 triple helical stability, showing that PBMCs can express sufficient quantities of this essential C7 post-translational modifying enzyme. Higher concentrations of C7 may be achieved from electroporating mRNA, and not naked DNA, into PBMCs. Similarly, improvements can be made to the transduction method to produce C7 expressing plasmablasts.

Electroporation-mediated delivery of mRNA encoding C7 is optimized. A research grade bulk flow-through electroporator is used to introduce mRNA encoding for C7 to re-program PBMCs to secrete C7 for in vitro and in vivo testing. All supernatant samples and animal studies are to be performed at Stanford. C7 will be purified from PCMC supernatant by C7 antibody (np185) affinity chromatography. Picograms of C7 produced per cell per day (PCD) will be assayed under in vitro conditions by SDS PAGE, followed by Coumassie Blue staining/densitometry, Western blot/densitometry and total amino acid analysis, and compared with rC7 currently being purified. The consistency on a batch per batch basis of the total expression of C7 in engineered PBMCs is determined. PBMC derived C7 is also analyzed by trypsin melting curve and circular dichroism to determine collagenous domain assembly/stability.

C7 purified from PBMCs will also be tested for binding to laminin-332 (its most important in vivo function) by adding samples of PBMC derived C7 to laminin-332 coated ELISA dishes. Next, following in vitro incubation of C7 expressing PBMCs with RDEB keratinocyte/fibroblast containing skin equivalents, assembly of PBMC derived C7 into anchoring fibrils in skin equivalent BMZ is analyzed by indirect immunofluorescent (IDIF) and immunoelectron microscopy (IEM) using specific antibodies to both the NC1 and NC2 ends of the C7 molecule (mAbs NP185 and LH24 respectively). Immunoelectron microscopy is performed. PBMCs are introduced in increasing numbers in order to determine the quantity needed for efficient C7 deposition over a ten-day time course.

In conjunction with in vitro studies above, RDEB keratinocyte/fibroblast containing skin equivalents are xenografted to immunodeficient mice, followed by IV administration of C7 overexpressing PBMCs. We vary the numbers of injected PBMCs over a range determined from the in vitro experiments to determine the minimal effective dose. Parameters to be assayed include clinical and histologic evaluation of dermal-epidermal separation of RDEB xenografts as well as incorporation of C7 into xenograft BMZ by IDIF.

Transmission and immuno-EM of skin biopsies will also be performed to assay number and quality of anchoring fibrils as well as proper localization and assembly of therapeutic C7 into these ultra-structural entities. Infiltration of genetically modified PBMCs into xenografts is quantified by FACS analysis of collagenase solubilized xenograft biopsy samples and IDIF of skin biopsies and compared with numbers of PBMCs in peripheral blood, spleen and draining lymph nodes. Samples are taken at one and two weeks following PBMC transfer and at monthly intervals thereafter up to 6 months, to determine persistence of efficacy and human C7/PBMC in vivo durability. Necroscopy and analysis of other tissues take place both to determine any systemic side effects of engineered PBMC delivery, and as part of toxicology studies.

To further preclinical development of C7 expressing plasmablasts and clinical development into a cell product bioengineered for sustained, systemic production of C7 as a functional cure for RDEB. The ISP™ cell culture system is used, and several non-viral vectors (i.e. Sleeping Beauty (SB) transposon system, zinc-finger nucleases (ZFNs), minicircles, CRISPR) to optimize protein production of C7 expressing plasmablasts.

### Example 2

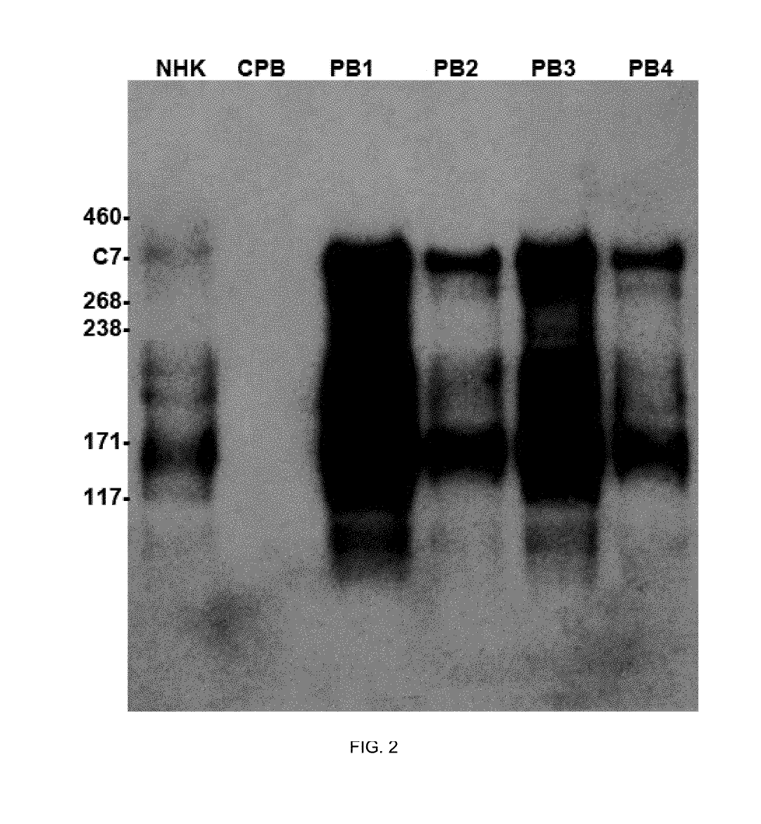

C7 overexpression in human peripheral blood mononuclear cells (PBMCs) and in CD19+ human plasmablast. While initial attempts to detect C7 in the supernatant from both PMBCs electroporated with naked DNA encoding C7 and ISP™ plasmablasts were negative, due to the large size of the COL71A expression cassette, the use of an optimized minimal backbone vector to improve the electroporation transduction efficiency was highly successful in expressing high levels of C7 in primary human plasmablasts. As can be seen in FIG. 2, unconcentrated supernatants of plasmablast cultures (PB1-4) show dramatically higher levels of C7 (perhaps tenfold if not more), compared to normal human keratinocytes (NHK). Taking into account that the keratinocyte medium was concentrated 100 fold prior to loading on the blot, this suggests that transfected plasmablasts may have expressed up to one thousand fold more C7 compared to normal keratinocytes. While keratinocytes (the major contributor of C7 to the BMZ), typically show significant in vitro degradation of their expressed C7 to a NC1 containing degradation fragment (see lower molecular weight band at approximately 171), plasmablast derived C7 appeared to showed less degradation and a greater proportion of intact full length C7, compared to keratinocyte derived C7. These results demonstrate that the stability of C7 derived from plasmablasts was at least as stable as C7 derived from keratinocytes.

The minimal backbone vector may be further refined, and the utility of dbDNA tested for transient expression of C7 from collection of peripheral blood monocytes (PBMCs), see Scott et al. (2015) Hum Vaccin Immunother. 11(8):1972-82. Testing may use autologous or allogenic MHC-matched primary B cells with RDEB skin graft equivalents. Non-donor matched healthy primary B cells are used for in vivo studies as final option. C7 expressing immune cells are assayed for prolyl 4 hydroxylase expression and activity, a key enzyme involved with C7 stability and assembly. C7 is purified from immune expressing cells by C7 antibody (NP185) affinity chromatography and is compared with well characterized human C7 purified at Stanford using a CHO mammalian expression system. Parameters to be evaluated include, approximate C7 expression levels of immune cells (by Western blot and SDS-PAGE analysis), total amino acid analysis, focusing on proline to hydroxyl proline ratio (to assess prolyl 4 hydroxylase activity), trypsin melting curves and circular dichroism (to determine triple helical stability) binding to BMZ ligands including type IV collagen and laminin-332, and reactivity with both NC1 and NC2 antibodies. In this way, structure and functionality of immune derived C7 are fully verified.

In vivo animal studies offer the opportunity to establish definitive protocols for assay detection of C7 secreted from ISP™ plasmablasts, to measure fibrotic changes of TGFβ driven pathways, to assess the impact of fibrosis, and to molecular characterize anchoring fibrils produced from ISP™ plasmablast C7 in RDEB skin graft equivalents. Towards this end, immunodeficient mice with RDEB skin xenografts are infused with an increasing dose range of C7 expressing ISP plasmablasts and injected cells are evaluated for persistence and biodistribution over time. Clinical observations are conducted at least twice daily for morbidity, mortality, and injury. Macroscopic and microscopic pathology of skin grafts and selected organs and tissues are evaluated during these intervals. Persistence of both the ISP™ plasmablasts themselves as well as their human C7 expression are evaluated. Samples of human RDEB skin xenografts, as well as mucosal tissues, lymphatic tissue, spleen, and bone marrow are evaluated by Fluorescence Activated Cell Sorting (FACS) analysis, immunofluorescence microscopy, and immuno-electron microscopy using antibodies specific to human C7 and other BMZ proteins as well as antibodies specific to human immune cells. Anchoring fibril morphology and number are assessed by electron microscopy. Time intervals of analysis following cell injections will be both short term (one week) as well as long term (up to six months). During these time periods, we will also analyze serum levels of C7 using a dual antibody sandwich ELISA system. This molecular analysis will be accompanied by a clinical-histologic evaluation of treated RDEB xenografts for evidence of correction of dermal-epidermal cohesion and blistering. Verification of correct epidermal differentiation will also be examined by immunofluorescence microscopy using a panel of differentiation markers in treated RDEB xenografts. In addition, fibrotic markers previously shown to be elevated in RDEB skin, including TGFβ, phospho-SMAD, collagen I, α smooth muscle actin, and picrosirius red, are assayed in order to determine whether C7 restoration to RDEB skin equivalents resulted in a reduction of dermal fibrosis.

1. A method for the treatment of epidermolysis bullosa (EB) in a subject, the method comprising:
administering to the subject an effective dose of a population of leukocytes that have been engineered to over-express human collagen VII (C7).

2. The method of claim 1, wherein the subject is a human.

3. The method of claim 1, wherein the subject is an immunodeficient mouse xenografted with keratinocyte/fibroblast containing skin equivalents.

4. The method of claim 2, wherein the human is suffering from a genetic defect in C7.

5. The method of claim 4, wherein the genetic defect is recessive dystrophic epidermolysis bullosa (RDEB).

6. The method of claim 1, wherein the leukocytes are peripheral blood mononuclear cells (PBMC).

7. The method of claim 6, wherein the PBMC are autologous to the subject.

8. The method of claim 6, wherein the PBMC are electroporated with mRNA encoding human C7.

9. The method of claim 1, wherein the leukocytes are B cells or plasmablasts derived therefrom.

10. The method of claim 9, wherein the B cells or plasmablasts derived therefrom are autologous to the subject.

11. The method of claim 9, wherein the B cells or plasmablasts derived therefrom are genetically modified by integration of an exogenous C7 coding sequence operably linked to a promoter active in the cell.

12. The method of claim 11, wherein the B cells or plasmablasts derived therefrom are further modified by integration of an exogenous prolyl-4-hydroxylase coding sequence operably linked to a promoter active in the cell.

13. The method of claim 11, wherein the cells are ex vivo differentiated plasmablasts.

14. An isolated population of engineered cells for use in the methods of claim 1.
